(19) United States
(12) Patent Application Publication
(10) Pub. No.: US 2010/0061944 A1
(43) Pub. Date: Mar. 11, 2010

(54) ORAL HYGIENE COMPOSITION COMPRISING MYRTLE

(75) Inventors: Zoe Marshall-Jones, Leicestershire (GB); Marie-Louise Baillon, Leicestershire (GB); Catherine Buckley, Leicestershire (GB)

Correspondence Address: FULBRIGHT & JAWORSKI, LLP, 1301 MCKINNEY, SUITE 5100, HOUSTON, TX, 77010-3095, US

(73) Assignee: Mars Incorporated, Mclean, VA (US)

(21) Appl. No.: 12/516,280

(22) PCT Filed: Nov. 27, 2007
(86) PCT No.: PCT/GB2007/004532

§ 371 Date: Aug. 14, 2009

(30) Foreign Application Priority Data
Nov. 27, 2006 (GB) 0623619.4

(51) Int. Cl. A61K 8/97; A61P 1/02

(52) U.S. Cl. 424/58

(57) ABSTRACT

The present invention relates to myrtle for use in oral health applications, an oral composition comprising myrtle, and the use of myrtle or the composition, in the improvement or maintenance of oral health in an animal, preferably through the reduction or control of dental plaque and/or alteration of the bacterial content of dental plaque, in the oral cavity of the animal. The invention also includes myrtle for use in the prevention or treatment of gingivitis in an animal. The invention also provides a method for improving or maintaining oral health in an animal.

### CROSS-REFERENCE TO RELATED APPLICATIONS

This application is a national stage filing of PCT/GB2007/004532 filed Nov. 27, 2007 which claims priority to Application No. 0623619.4 filed on Nov. 27, 2006 in the United Kingdom.

### TECHNICAL FIELD

The present invention relates to myrtle for use in oral health applications, an oral composition comprising myrtle, and the use of myrtle or the composition, in the improvement or maintenance of oral health in an animal, preferably through the reduction or control of dental plaque and/or alteration of the bacterial content of dental plaque, in the oral cavity of the animal. The invention also includes myrtle for use in the prevention or treatment of gingivitis in an animal. The invention also provides a method for improving or maintaining oral health in an animal

### BACKGROUND OF THE INVENTION

The need to maintain or improve oral health in an animal is of great importance. Poor oral health can lead to gum disease (gingivitis) and ultimately tooth loss, which can have severe effects on the wellbeing of the animal.

Poor oral health can be caused by a number of diseases and conditions. One of the most prevalent amongst cats and dogs is periodontal disease. Periodontal disease affects all cats and dogs at some stage during their lives. The aetiological agent in all cases of periodontal disease is plaque.

Current dietary methods for reducing or controlling plaque formation (and therefore associated conditions, such as gingivitis), in companion animals are usually mechanical means, such as hard chews or treats which act to scrape the plaque from the teeth, when chewed or consumed by the animal. The mechanical means rely on texture for their efficacy and a chewy rather than brittle texture is preferable to resist breakage of the means and therefore to also increase tooth cleaning time during chewing. Cats are less keen than dogs to chew for prolonged periods. Therefore products for various animals differ in texture to allow for these different preferences.

Textured toys may also be employed, to remove plaque mechanically from the surface of the teeth, without the animal ingesting any of the product that provides the textured surface.

However, the removal of plaque by mechanical means such as textured foodstuffs or toys relies upon the animal spending sufficient time chewing the mechanical means to scrape the plaque from the surface of the teeth. The amount of time required is difficult to assess and to monitor. In addition, plaque control on all tooth surfaces in the oral cavity is difficult to achieve via mechanical abrasion alone and certain teeth receive more efficient cleaning than others.

Plaque may also be removed or reduced by cleaning the teeth by brushing. However, owner compliance with toothbrushing is poor, with the result that very few dogs and cats receive a daily oral care regime of toothbrushing.

As an alternative to mechanical means for the removal of plaque, certain synthetic compounds such as chlorhexidine and triclosan can be used as antibacterial agents to reduce plaque. However, these compounds are broad spectrum antibacterial agents and, as such, may cause an imbalance in healthy gut microflora populations when ingested regularly. In addition, certain plaque bacteria have been associated with periodontal health and treatment with broad spectrum antibacterials would potentially kill these populations and would actually result in a less healthy oral microflora, leading to a reduction in oral health.

Accumulation of bacterial biofilms on the surface of a tooth can lead to gingivitis if not sufficiently addressed. Gingivitis is an inflammation of the gums caused by bacterial plaque that accumulates on the gum line. It can cause soreness, redness and bleeding of the gums.

An additional contributory factor to poor oral health is calculus. Since calculus cannot be removed by toothbrushing in normal cases, it accumulates on the tooth surface and irritates the gum tissue, giving rise to gingivitis. This is a further indication of poor or deteriorating oral health.

The addition of calculus formation inhibitors such as sodium tripolyphosphate to pet foodstuffs or human oral care products helps to prevent calculus accumulation. However, this does not address the bacterial community composition within the dental plaque that is contributing to the detrimental effects of periodontal disease on the oral health of the animal.

Therefore, there is a need for reducing the effects of dental plaque in an animal, in particular by natural methods, without relying solely on mechanical means or synthetic chemicals or compounds and without stressing the animal. Furthermore, there remains a need for the prevention and treatment of gingivitis in an animal.

### BRIEF SUMMARY OF THE INVENTION

Accordingly, the present invention provides myrtle for use in improving or maintaining oral health in an animal.

Myrtle (Myrtus communis) is a flowering plant in the family Myrtaceae, native to southern Europe and north Africa.

The inventors have unexpectedly found that myrtle is able to improve and/or maintain oral health in an animal.

Preferably, the myrtle improves or maintains the oral health of the animal by controlling or reducing dental plaque in the animal, by which it is meant that disease causing factors produced by the plaque and/or dental plaque is reduced or inhibited in the oral cavity of the animal.

Dental plaque is a mixed microbial community consisting of aerobic and anaerobic bacteria. Although plaque may vary between individuals the formation process can be broken down into three key events of (i) primary colonisation (adhesion); (ii) secondary colonisation (coaggregation); and (iii) maturation (virulence).

Plaque development begins with a tooth surface covered with a film of proteins and glycoproteins called the tooth salivary pellicle. Pioneer bacterial species adhere to molecules within the salivary pellicle, first forming a monolayer and subsequently pallisades of bacteria perpendicular to the tooth surface.

The microbe is held for a brief period by a weakly attractive force, during which time a number of specific adhesion mechanisms hold the cell close to the surface for a significant time period. These specific interactions may be a combination of lectin-like, electrostatic and hydrophobic interactions that in some instances could involve delicate structures called fibrils or fimbriae that project from the cell surface. Following this, initial attachment is rendered effectively irreversible by the production of extra-cellular polymers.

In humans streptococci are the most common primary colonisers making up between 47-52% of all bacteria adhering to the salivary pellicle.

During and after this initial phase, secondary colonisation by a variety of bacteria occurs leading to a large increase in bacterial diversity. Foremost among the events of secondary colonisation is the process of coaggregation whereby the primary colonisers now act as the substrate for colonisation.

Coaggregation has been described as ‘the recognition between surface molecules on two different bacterial cell types so that a mixed cell aggregate is formed’. It has also be described as ‘the adherence among partner cells in a suspension’.

Coaggregation is a highly specific process that takes place between specific bacterial ‘partners’. Each strain has its own set of partners and mechanisms of cell-cell recognition. Groups of strains also exist which are able to coaggregate with several other strains. Based on human studies, one such organism that dominates these later colonisers is Fusobacterium nucleatum, which is a dominant organism in mature dental plaque.

Coaggregation is known to play an important role in human plaque formation. Coaggregation between different strains of canine oral bacteria has been determined in vitro suggesting a similar role for this behaviour in dental plaque formation and development in other animals.

At some point during the development of the plaque biofilm, the rate of change in the overall composition slows. The point at which this happens is currently unknown, although it is thought to take several days for the biofilm to reach this state.

In human plaque, a succession of bacterial species occurs as Gram-positive cocci and rods are progressively replaced by Gram-negative filamentous and flagellated organisms. The maturing biofilm also tends to become increasingly anaerobic as it increases in depth.

It is at this point that the biofilm can be said to have reached a climax community, where a number of the bacteria are reliant on others within the biofilm for their survival. It is during this phase that many organisms associated with periodontal disease are present. These bacteria produce a number of compounds that are the causative factor of periodontal disease, such as proteases and haemolysins. Proteases, in particular trypsin, are reported to have a host of abilities, including the ability to degrade immunoglobulins, inactivate cytokines and their receptors, degrade host tissues and promote bleeding in the oral cavity. The bacteria of the plaque is known as the plaque biomass.

Pathogenic bacteria, such as Peptostreptococcus are often present in dental plaque, as well as black pigmenting anaerobes, such as Porphyromonas, Bacteroides and Prevotella, all of which are thought to contribute to disease states.

The myrtle of the invention is useful for inhibiting the formation of such biofilms and/or inhibiting the detrimental activities of the biofilm and therefore improving or maintaining oral health by controlling or reducing dental plaque in an animal. The myrtle of the invention is also provided for the prevention or treatment of gingivitis in an animal.

By reducing the level of pathogenic bacteria in the biofilm, the health of the dental plaque is improved. Thus, the myrtle of the invention is useful in altering the bacterial content of the plaque, preferably by reducing the pathogenic bacterial content of the plaque in the oral cavity of an animal. The myrtle may also promote the healthy bacteria of the plaque. The myrtle of the invention is useful in improving the health of the dental plaque present in the oral cavity of an animal.

The myrtle of the invention preferably reduces the level of inflammatory proteases and/or black pigmenting anaerobes in dental plaque in an animal. These are key disease causing agents that are found in dental plaque.

Most preferably, myrtle inhibits or reduces pathogenic bacteria in dental plaque, which preferably includes Peptostreptococcus sp.

The myrtle of the invention is suitable for any animal including a human. However, in a preferred embodiment the animal is a companion animal or a human. By companion animal it is meant any animal that is kept as a pet, which includes a cat, a dog, a horse, a rabbit, or a guinea pig. Preferably, the composition is for a cat or a dog or a human.

The myrtle variety is preferably Myrtus communis, which is also known by several other names including Myrtus baetica, Myrtus italica, Myrtus romanifolia, Myrtus macrofilia, Myrtus littoralis, Myrtus minima. The skilled person understands that other names are used to refer to this species of myrtle including Myrtus baetica var. vidalii, Myrtus communis var. christinae, Myrtus communis var. eusebii, Myrtus communis var. gervasii, Myrtus italica var. briquetii, Myrtus italica var. petri-ludovici, Myrtus communis var. acutifolia, Myrtus communis var. angustifolia, Myrtus communis var. baetica, Myrtus communis var. belgica, Myrtus communis var. mucronata, Myrtus communis var. romana, Myrtus major Garsault, Myrtus minor Garsault, Myrtus acuta Mill, Myrtus baetica Mill, Myrtus belgica Mill, Myrtus italica Mill, Myrtus minima Mill, Myrtus littoralis Salisb, Myrtus macrophylla, Myrtus macrophylla Myrtus romanifolia, Myrtus communis subsp. Mucronata, Myrtus media, Myrtus romana Hoffmanns, Myrtus angustifolia, Myrtus buxifolia Raf, Myrtus lanceolata Raf., Myrtus latifolia Raf., Myrtus oerstedeana, Myrtus sparsifolia, Myrtus veneris Bubani, Myrtus communis var. acuminata, Myrtus communis var. italica (Mill.), Myrtus communis var. lusitanica, Myrtus borbonis Sennen, Myrtus acutifolia (L.), Myrtus augustinii, Myrtus baui, Myrtus briquetii, Myrtus christinae, Myrtus communis var. balearica, Myrtus communis var. foucaudii, Myrtus communis var. grandifolia, Myrtus communis var. joussetii, Myrtus communis var. neapolitana, Myrtus eusebii, Myrtus gervasii, Myrtus josephi, Myrtus mirifolia, Myrtus petri-ludovici, Myrtus rodesi, Myrtus theodori, and Myrtus vidalii

The myrtle of the invention can be the whole plant or part thereof. It may be the root, bark, stem, leaf, sap, flower or any combination thereof. The myrtle may be dried, crushed, ground or shredded. Preferably, the myrtle to be used is myrtle leaf.

Additionally or alternatively an extract of myrtle may be used. Suitable extracts include methanol extract, ethanol extract, chloroform extract or water extract. Any other suitable extract may be used, as understood by the skilled person.

A second aspect of the invention provides an oral composition comprising myrtle.

The myrtle may comprise between 0.1%-20% by weight of the composition, more preferably 1-15% by weight, more preferably 3-10% by weight, or 1, 2, 3, 4, 5, 6, 7, 8, 9, or 10% by weight. Most preferably, the myrtle comprises about 3% by weight of the composition.

The composition may comprise myrtle as the only active ingredient with respect to the improvement or maintenance of oral health. Alternatively, the composition may comprise myrtle as part of a cocktail including one or more further oral health improving or maintaining, or plaque reducing or controlling components.

Hereinafter in this text, the term “oral composition” covers all compositions that come into contact with the oral cavity, preferably the surface of a tooth of an animal, including a foodstuff, diet and supplement. Any of these forms may be solid, semi-solid or liquid. The composition may be a paste or a gel.

The composition may be in the form of a supplement to be added to any foodstuff that does not contain sufficient levels of myrtle to improve or maintain oral health including prevention or treatment of gingivitis, or to control or reduce dental plaque in an animal, by way of reduction or inhibition of disease causing factors and/or biomass in the plaque.

The concentration of myrtle in the supplement may be used in addition to the animal's main diet or foodstuff. This can be done by including a quantity of the supplement with the animal's diet or by additionally feeding the animal a quantity of the supplement. The supplement can be formed as a foodstuff with extremely high levels of the myrtle composition of the invention, which requires dilution before feeding to the animal. The supplement may be in any form, including solid (e.g. a powder), semi-solid (e.g. a food-like consistency/gel), a liquid, a paste or alternatively, it may be in the form of a tablet or capsule. The liquid can conveniently be mixed in with the food or fed directly to the animal, for example via a spoon or via a pipette-like device. The supplement may be high in one or more components of the invention or may be in the form of a combined pack of at least two parts, each part containing the required level of one or more component.

Preferably the myrtle or a composition comprising myrtle is incorporated into a commercial petfood product composition or a commercial dietary supplement composition. The petfood product may be a dry, semi-dry, a moist or a liquid (drink) product. Moist products include food which is sold in tins or foil containers and has a moisture content of 70 to 90%. Dry products include food which have a similar composition, but with 5 to 15% moisture and presented as biscuit-like kibbles. When the composition comprises a diet, foodstuff or supplement, it is preferably packaged. In this way the consumer is able to identify, from the packaging, the ingredients in the food and identify that it is suitable for the animal in question. The packaging may be metal (usually in the form of a tin or flexifoil), plastic, paper or card. The amount of moisture in any product may influence the type of packaging which can be used or is required.

The composition according to the present invention encompasses any product which an animal may consume in its diet. Thus, the invention covers standard food products for humans or other animals, as well as pet food snacks (for example snack bars, biscuits and sweet products). The composition may be a cooked product. It may incorporate meat or animal derived material (such as beef, chicken, turkey, lamb, blood plasma, marrowbone etc, or two or more thereof). The composition alternatively may be meat free (preferably including a meat substitute such as soya, maize gluten or a soya product) in order to provide a protein source. The composition may contain additional protein sources such as soya protein concentrate, milk proteins, gluten etc. The composition may also contain a starch source such as one or more grains (e.g. wheat, corn, rice, oats, barely etc) or may be starch free. A typical dry commercial dog and cat food contains about 30% crude protein, about 10-20% fat and the remainder being carbohydrate, including dietary fibre and ash. A typical wet, or moist product contains (on a dry matter basis) about 40% fat, 50% protein and the remainder being fibre and ash. The composition of the present invention is particularly relevant for a foodstuff as herein described which is sold as a diet, foodstuff or supplement for a cat, a dog or any other companion animal or a human.

In the present text the terms “domestic” dog and “domestic” cat mean dogs and cats, in particular Felis domesticus and Canis domesticus.

The composition may be applied to or incorporated within a chew or treat which the animal may consume in addition to a main meal foodstuff. The composition may be provided as a coating on or incorporated within a main meal foodstuff.

Alternatively, the composition may be a liquid, gel, paste or the like which may be applied as a coating to a non-consumable product, such as a toy for an animal. The composition may be incorporated within the product. When the animal chews the toy, the composition comes into contact with some or all of the oral cavity of the animal and improves or maintains the oral health of the animal.

When the composition is incorporated within or coated onto a chewy or hard product, the additional benefit of improving or maintaining the oral health of the animal by removing plaque through the mechanical action of the product against the teeth of the animal is achieved, as well as by the action of the myrtle in the composition.

The inhibition of certain plaque biofilm forming bacteria by myrtle results in the control or reduction of dental plaque in an animal by the reduction of the bacterial content of the dental plaque.

The composition may be used for an animal with any level of oral health in order to improve or maintain oral health in the animal.

The composition may be used for an animal with good or acceptable oral health in order to maintain oral health. The composition in this case may control dental plaque formation and minimise the destructive effects of certain plaque bacteria on the periodontal health of the animal.

Alternatively, the composition may be used for an animal with poor oral health in order to improve the oral health of the animal. The improvement of oral health may be by way of the control of the further accumulation of dental plaque and slow the progression of the disease into the severest stages. It may also reduce dental plaque already present on the surface of the teeth of the animal. In cases of moderate to severe periodontal disease, the animal may require veterinary and/or dental attention prior to using the composition in order to achieve oral health benefits and reduce the frequency of future veterinary and/or dental intervention.

The composition is an oral composition. By oral composition it is meant that during use the oral cavity of the animal is exposed to the composition, and preferably the composition has direct contact with the surface of a tooth of the animal. Most preferably, the surface of a tooth is directly contacted with the myrtle of the composition.

Such an oral composition can include toothpaste, mouthwash or any other such gel, liquid or paste. The oral composition may be a foodstuff, as previously defined.

A third aspect of the invention provides the use of myrtle in the manufacture of a composition for the improvement or maintenance of oral health in an animal. Preferably, the oral health is improved or maintained by the control or reduction of dental plaque in the animal including reduction and/or inhibition of disease causing factors, biomass or pathogenic bacteria. The use of myrtle in the manufacture of a composition for the prevention or treatment of gingivitis is also provided.

The invention, as a fourth aspect, also provides a method for the improvement or maintenance of oral health in an animal comprising administering to the animal myrtle or a composition of the second aspect. Preferably, the method improves or maintains the oral health of the animal by the reduction or control of dental plaque in the animal, as previously defined.

In the method of the fourth aspect, the oral cavity of the animal is exposed to the composition, by way of consumption of the composition through its inclusion in a foodstuff, or by way of a coating comprising the composition on a toy which the animal chews.

Preferably, the method is for use in an animal susceptible to poor oral health or dental plaque, gingivitis or periodontal disease.

The composition may be administered to an animal with poor oral health to reduce the amount of dental plaque or factors contained therein, and then continued feedings may be carried out to control, reduce or inhibit the formation of further dental plaque or any one or more of the factors contained therein. The animal may require veterinary and/or dental treatment before or during use of the composition to remove calculus deposits in order to see a beneficial effect of the myrtle or the composition.

By poor oral health is meant the presence of a number of indicators of this status including calculus and plaque accumulation, gingivitis, oral malodour, presence of gingival recession and/or periodontal pockets, as will be appreciated by the skilled person.

All features of each aspect of the invention relate to all other aspects mutatis mutandis, as appreciated by the skilled person.

### BRIEF DESCRIPTION OF THE DRAWINGS

The invention will now be described with reference to the following non-limiting examples and figures, in which:

FIG. 1 shows the effect of myrtle on facultative anaerobes cultured from treated biofilms expressed as a percentage of untreated controls. Untreated CFU (100%)=4.05×10̂7/ml;

FIG. 2 shows the effect of myrtle on fastidious anaerobes cultured from treated biofilms expressed as a percentage of untreated controls. Untreated CFU (100%)=2.96×10̂7; and

FIG. 3 shows the effect of myrtle on Peptostreptococcus stomatis colonies cultured from treated single species biofilms expressed as a percentage of untreated controls. Untreated CFU (100%)=1.34×10̂7.

### DETAILED DESCRIPTION OF THE INVENTION

### Examples

Myrtle was tested for its ability to control or reduce dental plaque in an animal by way of the following in vitro experiments. Supragingival plaque was obtained from dogs and various assays were carried out, as described below, to determine whether myrtle has the ability to improve or maintain oral health in an animal.

### Example 1

Initial assays were set up to determine whether myrtle is suitable for use in an animal for improving or maintaining oral health.

These assays include the ability to inhibit adhesion of plaque forming bacteria, inhibit growth of oral bacteria, inhibit protease production in oral bacteria and inhibit haemolysis caused by oral bacterial strains.

Myrtle inhibited adhesion by up to 100%, growth by up to 93%, protease production by up to 57% and showed the ability to inhibit haemolysis in 5 out of 8 oral bacterial strains tested.

These results showed that myrtle has the ability to inhibit undesirable oral bacteria and therefore it was tested in further assays for its ability to maintain or improve oral health in an animal.

### Example 2

### Assay Inoculum: Plaque and Saliva Sampling from Dogs

The assay requires fresh supragingival canine dental plaque and saliva for inoculation. The inoculum consists of pooled dental plaque and unfiltered saliva sampled from a group of 14 dogs, varying in age, breed and oral health status.

The plaque and saliva were resuspended in artificial saliva to form the inoculum of approximately 15% plaque and 30% saliva.

### Assay Set-Up

The plate biofilm assay (PBA) utilises a 24 well plate format in which biofilms, representative of canine dental plaque, are grown on hydroxyapatite (HA) discs. Prior to being introduced to the 24 well assay plate, each HA disc is preconditioned for 2 hours in a solution of 50% filter sterilised canine saliva in artificial canine saliva. The preconditioning step stimulates the formation of a salivary pellicle on the HA disc surface. Following preconditioning, each HA disc is placed individually into a well on the 24 well plate. The inoculum is divided into two equal aliquots and the active added to one aliquot at the appropriate concentration. The other aliquot represents the control (no active). A 1 ml inoculum is added to each well and the assay plate incubated aerobically with shaking at 38° C. for 48 hours. After 24 hours and 30 hours, the discs are transferred into fresh artificial saliva containing the active at the appropriate concentration as before. Biofilm-covered HA discs are removed from the assay plate for analysis after 48 hours. Each HA disc, with the exception of those being used for biomass quantification, is placed into 500 μl PBS and vortex mixed for 30 seconds to remove biofilm growth from the disc into solution. Biofilm suspensions are then used for analysis. Biofilm-covered HA discs that are being used for biomass quantification are removed from the 24 well assay plate and used directly in the crystal violet assay.

### Example 3

### Myrtle Extracts Tested in the PBA

A methanol extract of myrtle was used for testing in the canine PBA. Extractions were performed as described previously.

The raw botanical of myrtle leaf was tested against clove (dried flower buds), parsley (leaf) and eucalyptus (leaf) in the canine PBA at 500 μg/ml and 5000 μg/ml. Myrtle shows an improved performance over parsley and eucalyptus in protease and biomass inhibition at both 500 μg/ml and 5000 μg/ml. Myrtle performs as well as clove in black pigmenting colony and protease inhibition at 5000 μg/ml.

In addition, chlorhexidine (Lloyds Pharmacy) was included as the gold standard reference or positive control. However, chlorhexidine is undesirable for use in animal compositions since it is a synthetic chemical and may have potential toxic effects as it is a chemical used in its purest form.

### Example 4

### Biofilm Measures

The following analyses were used to assess the biofilms produced in the canine PBA and the effects of myrtle and the non-botanical compounds on biofilm development:

Biomass quantification (crystal violet assay)

Protease activity

Bacterial viable counts

A brief description of each assay is given below.

### Biomass

The total amount of biofilm grown on the HA discs was quantified using the crystal violet staining method. Biomass was represented as being directly proportional to the OD reading at 595 nm (OD₅₉₅) of the samples compared to controls. Results were expressed as the reduction in OD₅₉₅ seen in active-treated samples compared to no active controls, reflecting the effect of the active treatment on the amount of biofilm growth on the disc.

Myrtle reduced biomass by 59.5%.

### Protease Activity

Trypsin-like protease activity was measured using the liquid BAPNA assay, a colourimetric assay in which the amount of trypsin present in a sample is directly proportional to the intensity of the colour developed. Samples were quantified against a trypsin standard curve and results expressed as the percentage inhibition of protease activity in active-treated samples compared to controls.

Myrtle reduced protease production by 74.34%

### Bacterial Counts

Viable numbers of bacteria were quantified using Columbia blood agar plates supplemented with haemin and menadione. Aerobes were counted after incubation for 2 days and anaerobes, including black pigmenting colonies (BPC), were counted after incubation at appropriate conditions for 9 days. Plate counts are expressed as colony forming units (cfu) per ml and differences between control and active plates are expressed in logs.

Myrtle reduced plate counts of black pigmenting bacterial colonies by 3.75 logs, compared to controls. This particular group of bacteria are thought to be important in periodontal disease.

### Example 5

### Statistical Analysis of Data

Each sample was repeated 5 times within the assay. Unless otherwise stated, all extracts were tested in the assay at a concentration of 500 μg/ml. For each sample, all of the values obtained were logged and the means calculated from the log values.

A 2-tailed t-test with unequal variance was then performed. An unequal variance analysis was selected as the individual analyses were independent i.e. the measures were not comparable to one another. For each data set, p values were obtained and these gave an indication of the reproducibility of the data.

### Results

A table summarising how myrtle performed in the tests is set out below:

TABLE 1

| | Aerobe | Anaerobe | BPC | | |
|---|---|---|---|---|---|
| | (Log 10 | (Log 10 | (Log 10 | Protease | Biomass |
| Common name | reduction) | reduction) | reduction) | (% reduction) | (% reduction) |
| Chlorhexidine | 2.87 | 2.48 | 2.74 | 95.76 | 94.40 |
| Myrtle leaf | 0.05 | −0.10 | 3.75 | 75.34 | 59.50 |
| Orthosiphon | −0.09 | 0.02 | 2.59 | 24.53 | 14.60 |
| Tepezcohuite | 0.25 | −0.42 | −0.51 | 80.25 | −27.40 |

As can be seen, Myrtus communis significantly reduced black pigmenting colony counts and had a significant inhibitory effect on protease and biomass.

### Example 6

### Testing of Raw Material

The raw plant material of myrtle was also tested in the Plate Biofilm Assay, as well as the extracts described above. The raw plant material was prepared through a 250 μm pore size sieve and was tested at 5000 μg/ml in the assay. The raw material was as effective at inhibiting biofilm formation as the previously tested extracts.

### Example 7

### Inhibition of Human Plaque

Myrtle leaf powder was tested for inhibition of biofilm formation in a human form of the Plate Biofilm Assay. The final concentration of each test agent was 250 μg/ml. Tests were repeated five times in separate assays.

Hydroxyapatite discs were incubated in 20% pooled human saliva for 2 hours at room temperature. An amount of 10 ml of pooled human saliva was collected and combined with plaque inoculum scraped from the tooth surface of human volunteers. The inoculum was added to the 20% pooled saliva at a ratio of 1:3 (v/v) and 1.33 ml of the resulting suspension was combined with 2.0 ml artificial saliva (Pratten et al., 1998) and 0.175 ml of the appropriate test agent (Myrtus communis, Uncaria tormentosa, Orthosiphon spicatus, parsley or eucalyptus) at a concentration of 5 mg/ml in sterile water or water (as a negative control to which each test agent was compared). Parsley and eucalyptus were used as positive controls, as they are each well known natural ingredients in oral health products due to their positive effect on oral health.

Triplicate aliquots of each solution (1 ml) were placed in individual wells of a sterile 24 well plate with a single saliva coated hydroxyapatite disc. The discs were incubated for 1 hour at 37° C. in anaerobic conditions (10% H₂, 10% CO₂, 80% N₂), allowing the growth of obligate anaerobes that are found in the sub-gingival recesses associated with periodontitis. This was followed by 24 hours incubation at 37° C. in aerobic conditions.

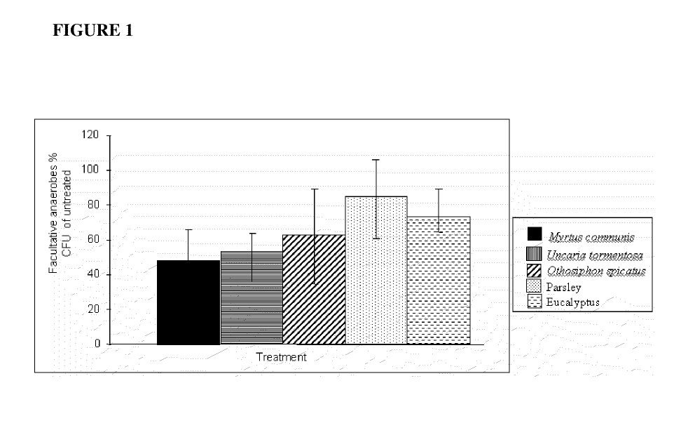

Biofilms were dispersed, serially diluted and then plated onto CBA (+ hemin, menadione) and incubated anaerobically or onto BHY and incubated aerobically. Colonies were counted after 24-48 hours. The results are shown in FIG. 1, where it can be seen that Myrtle (Myrtus communis) inhibited the numbers of facultative anaerobic bacteria in human plaque biofilms in vitro compared to untreated (water) control. Surprisingly, myrtle was more effective at reducing levels of these organisms than parsley and eucalyptus, known oral health promotors.

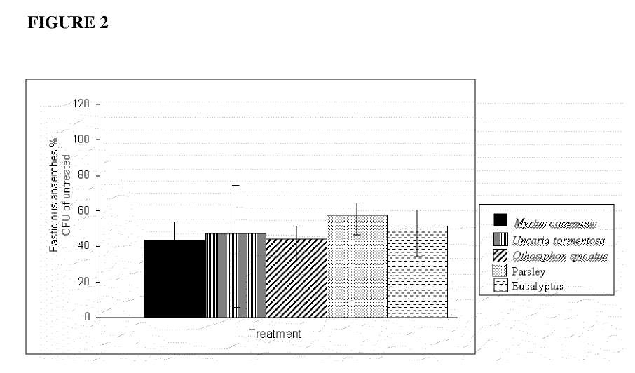

Fastidious anaerobe numbers were also counted, and were also seen to be reduced compared to untreated controls, as shown in FIG. 2. It was also unexpectedly found that myrtle performed better than parsley and eucalyptus in inhibiting fastidious anaerobes.

Myrtle leaf powder was also tested for inhibition of Peptostreptococcus stomatis growth in artificial saliva under plaque biofilm assay conditions described above (final concentration of the agents was 0.25 mg/ml). Colonies were counted after 24 hours growth in anaerobic cabinet.

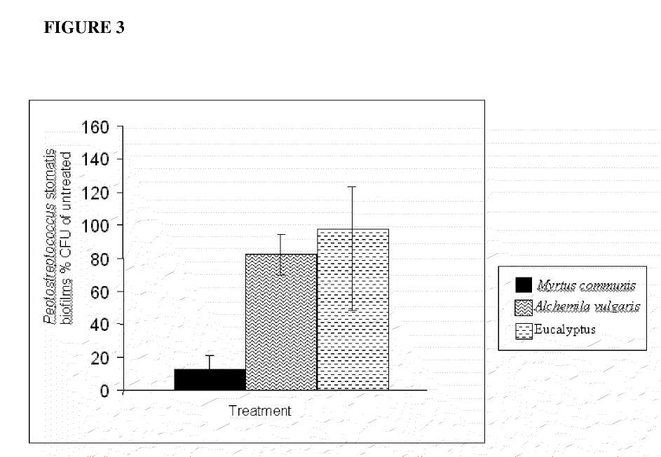

Myrtle leaf treatment substantially reduced bacterial numbers in Peptostreptococcus biofilms compared to both untreated controls and those treated with eucalyptus leaf powder (FIG. 3). Peptostreptococcus are pathogenic bacteria, known to be associated with gingivitis, periodontitis and oral health problems.

### Example 8

Various product applications require survival of the raw material activity following exposure to temperatures up to 120° C. To test this, the raw myrtle leaf was heated to 120° C. for 10 minutes and its activity tested in the Plate Biofilm Assay compared with non heat-treated controls.

Heat treatment of Myrtus communis, as described above, does not affect its performance. Heat-treated Myrtus communis reduces biomass by 94.4%, compared to 97.7% in the unheated control. Protease is completely inhibited (100%) in both the heat-treated and non-heated control.

### Example 9

To assess product acceptance, myrtle leaf was included in a 25 g chew format at a level of 3% and fed to miniature schnauzers, cocker spaniels and Labradors in a crossover study with three other chew types. A chew was given once per day for 4 days and a washout period of 3 days was allowed before commencing the next feeding phase. When compared with the standard chew containing no myrtle, acceptance of the myrtle chew was similar in all dogs.

### Example 10

To assess the efficacy of myrtle for maintenance and improvement of oral health in companion animals myrtle leaf was included in a chew format at a level of 2.65% and fed to miniature schnauzers (17 g chew), cocker spaniels (25 g chew) and Labradors (40 g chew). The effect of the myrtle composition on oral health compared to that resulting from the standard chew, a second dental chew and to a dry kibble base diet was assessed. Thirty-two healthy adult dogs were assigned to one of 4 groups with a total of twelve Labrador Retrievers, twelve Cocker Spaniels and 8 Miniature Schnauzers. Animals were randomly assigned to groups within weighted blocks to ensure breed, sex and approximate age matching.

Animals lived in pairs in environmentally enriched two roomed housing with 24 h access to the outside and free access to exercise paddocks during daylight hours. Full animal welfare considerations were in place. The study was approved by the WALTHAM Centre for Pet Nutrition ethical review committee, in accordance with the UK Home Office Animals (Scientific Procedures) Act 1986. Dogs were socialized and walked daily and fresh water was available at all times. The animals were fed once daily at energy levels (calorific values) that were required in order to maintain bodyweight

The study utilised a four phase Latin square design with repeated measures. In this clean tooth model, the dogs were given a dental scale and polish at day 1 and received a standard commercial dry kibble diet and daily tooth brushing for two weeks (baseline phase) to reduce gingivitis to baseline levels. Gingivitis scores and removal of any accumulated dental deposits was then undertaken through a second dental scale and polish, following which animals received the same commercial dry kibble base diet plus test product for a five week period prior to repeated gingivitis scoring as well as measurement of plaque and calculus deposits. Group 1 (control animals) were maintained on the base diet only; group 2 in addition to base diet received a daily standard dental chew; group 3 received the same dental chew with 2.65% Myrtle leaf daily and group 4 received an alternative chew format not containing the active ingredient (data not shown for alternative chew format).

Following the end of phase 1 as described above each group transferred to the next dietary regime and repeated measures were taken in each subsequent phase until all of the dogs had received all of the diets. Gingivitis, plaque and calculus scores were assessed using the modified Logan & Boyce technique (Hennet et al., 2006) at the beginning and completion of the 5-week test period.

The following teeth were used for assessments of oral health.

Maxilla: I3 (103,203), C (104,204), P2 (106,206), P3 (107,207), P4 (108,208), and M1 (109,209).

Mandible: C (304,404), P2 (306,406), P3 (307,407), P4 (308,408), and M1 (309, 409).

Gingivitis was measured along the buccal surface at the gingival sulcus. The gingiva were divided into thirds (mesial, buccal and distal) and a score was given to each third. Tooth scores were calculated as the mean score of the three sections and total scores as the mean of all of the teeth assessed.

Criteria

0-No gingivitis, pink (or pigmented) healthy gingiva no inflammation no bleeding on probing

1-Very mild gingivitis (red, swollen but no bleed on probing)

2-Mild gingivitis (red, swollen and delayed bleeding on probing)

3-Moderate gingivitis (red, swollen and immediate bleeding on probing)

4-Severe gingivitis (ulceration, spontaneous hemorrhage, profuse bleeding on probing)

Plaque was disclosed on the buccal surface of the teeth by applying an undiluted disclosing solution (Erythrosin) and immediately rinsing with water. Each of the scored teeth was assessed for coronal and gingival plaque levels according to Hennet et al. (2006). The two halves of the tooth crown (coronal and gingival) were successively assessed for plaque coverage and thickness was assessed on the uncovered part using a dye reference solution colour palette for the thickness assessment. The shade that is closest to that on the disclosed surface was designated as the thickness score. Scores on both the coronal and gingival sections were totalled to give a total tooth score. The means of all tooth scores provided the total mouth score.

Calculus was air-dried and a dental probe was used to gently verify the visual appearance of coverage and thickness. A coverage and thickness score was given for the gingival and coverage and thickness scores for gingival and coronal areas of the tooth were multiplied to give a total tooth score, mouth scores were calculated as for plaque coverage.

In addition to the clinical oral health assessments a supra-gingival plaque sample was scraped from the teeth of each dog during week 2 of the test phase. This was followed by thoroughly tooth brushing each dog to ensure any remaining dental deposits were removed.

Analyses were undertaken on the response variables plaque, gingivitis and calculus using a general linear model (GLM) to test for treatment, phase and sequence effects. Significance levels were reported along with estimates of treatment effects. Data were coded in Excel workbooks and analysed using proprietary statistical software routines (Minitab Verion 14).

### Results

The dental chews containing myrtle leaf powder significantly (P=<0.05) reduced mean gingivitis levels compared to base diet while the standard chew did not show significant reductions compared to base diet. Dogs being fed dental chews containing myrtle resulted in a mean gingivitis score below those observed at baseline following two weeks tooth brushing.

Mean plaque (P=<0.1) and calculus (P=<0.05) scores were reduced compared to standard diet but were slightly higher than those observed for dogs receiving the standard dental chew.

TABLE 2
Effect of the three dietary routines on clinical measures of oral health

| | | Mean Gingivitis | | |
|---|---|---|---|---|
| | Mean Gingivitis | Score | | |
| | Score | (end of test phase | Mean Plaque | Mean Calculus |
| Diet | (end of test phase) | minus baseline) | Score | Score |
| Standard Diet | 1.31 | 0.12 | 9.08 | 1.12 |
| Standard diet and | 1.24 | 0.01 | 8.04 | 0.72 |
| dental chew | | | | |
| Standard diet and | 1.20 | −0.01 | 8.27 | 0.79 |
| dental chew with | | | | |
| Myrtle | | | | |

### REFERENCES

Hennet P, Servet E, Salesse H, Soulard Y: Evaluation of the Logan and Boyce Plaque Index for the Study of Dental Plaque Accumulation in Dogs. Res Vet Sci, 80, 175-180, 2006.

Pratten, J., Smith, A. W. and Wilson, M. (1998) Response of single species biofilms and microcosm dental plaques to pulsing with chlorhexidine. J Antimicrob Chem 42, 453-459.

1. A method of using myrtle comprising: the use of myrtle in the maintenance or improvement of oral health in an animal.

2. The method according to claim 1, the use of myrtle in the prevention or treatment of gingivitis in an animal.

3. The method according to claim 1, wherein the myrtle reduces, inhibits or controls disease causing factors produced by plaque and/or dental plaque in the oral cavity of the animal.

4. The method according to claim 1, wherein the myrtle alters the bacterial content of dental plaque in the oral cavity of the animal.

5. The method according to claim 4, wherein the myrtle alters the bacterial content by reducing the bacterial content.

6. The method according to claim 1, wherein the myrtle improves the health of dental plaque present in the oral cavity of the animal.

7. The method according to claim 1, wherein the myrtle inhibits or reduces inflammatory proteases and/or pathogenic bacteria in dental plaque.

8. The method according to claim 7, wherein the pathogenic bacteria include black pigmenting anaerobes.

9. The method according to claim 7, wherein the pathogenic bacteria include Peptostreptococcus.

10. The method according to claim 1, wherein the myrtle is Myrtus communis.

11. The method according to claim 1, wherein the animal is a cat, a dog or a human.

12. An oral composition comprising myrtle.

13. The composition according to claim 12, for use as set out in claim 1.

14. The composition according claim 12, wherein the myrtle is present at a concentration of from 0.1% to 20% by weight of the composition.

15. The composition according to claim 12, wherein the composition is a foodstuff.

16. The manufacture of a composition comprising the step of: using myrtle in the manufacture of a composition for the improvement or maintenance of oral health in an animal.

17. The manufacture of a composition according to claim 16, wherein the composition is for the prevention or treatment of gingivitis in an animal.

18. The manufacture of a composition according to claim 16, wherein the composition reduces or controls dental plaque in the animal.

19. A method of maintaining or improving oral health in an animal comprising administering to the animal an effective amount of myrtle or the composition according to claim 12.

20. (canceled)

21. (canceled)

22. (canceled)
